(19) United States
(12) Patent Application Publication
(10) Pub. No.: US 2021/0299485 A1
(43) Pub. Date: Sep. 30, 2021

(54) HEATED MASK

(71) Applicant: TRAMEC TERMICO TECHNOLOGIES LLC, Elk Grove Village, IL (US)

(72) Inventor: Michael M. Cubon, Park Ridge, IL (US)

(21) Appl. No.: 17/215,482

(22) Filed: Mar. 29, 2021

Related U.S. Application Data
(60) Provisional application No. 63/000,671, filed on Mar. 27, 2020.

(51) Int. Cl. A62B 9/00; A41D 13/11; A41D 13/005; A61L 9/16; A62B 18/08; A62B 18/02; A62B 7/00; H05B 1/02; H05B 3/36

(52) U.S. Cl. CPC A62B 9/003; A41D 13/113; A41D 13/0051; A61L 9/16; A62B 18/08; A62B 18/025; H05B 2203/02; H05B 1/0252; H05B 1/0202; H05B 3/36; A41D 2400/10; A62B 18/084; H05B 2203/016; A62B 7/00

(57) ABSTRACT

A mask includes a support member that is configured to extend across at least a portion of a face of a user and a heater connected to the support member.

### RELATED APPLICATIONS

This application claims priority from U.S. Provisional Application Ser. No. 63/000,671, filed Mar. 27, 2020, the subject matter of which is incorporated herein by reference in its entirety.

### FIELD OF THE INVENTION

The present invention relates to a heated mask and, more specifically, to a heated mask with a polymeric positive temperature coefficient (PTC) heater.

### BACKGROUND OF THE INVENTION

A virus may be spread from one person to another via respiratory droplets produced during coughing and/or sneezing. Disease 2019 (COVID-19) is an infectious disease caused by severe acute respiratory syndrome coronavirus 2 (SARS-CoV-2). The disease was first identified in 2019 in Wuhan, China, and has since spread globally, resulting in the 2019-20 coronavirus pandemic. Common symptoms include fever, cough, and shortness of breath. Muscle pain, sputum production and sore throat are less common. While the majority of cases result in mild symptoms, some progress to severe pneumonia and multi-organ failure. Patients with preexisting conditions, including hypertension, diabetes mellitus and cardiovascular disease, are most at risk and represent the vast majority of deaths. The infection is typically spread from one person to another via respiratory droplets produced during coughing and/or sneezing. Time from exposure to onset of symptoms is generally between two and 14 days, with an average of five days. The standard method of diagnosis is by reverse transcription polymerase chain reaction (rRT-PCR) from a nasopharyngeal swab. The infection can also be diagnosed from a combination of symptoms, risk factors and a chest CT scan showing features of pneumonia.

### SUMMARY OF THE INVENTION

The present invention is directed to a mask to inhibit, slow, deactivate and/or kill a virus. Additionally, the mask may help prevent a virus's entry into the respiratory system. More specifically, the invention is directed to a mask to inhibit, slow, deactivate and/or kill the Covid-19 virus since it may be sensitive to heat and can be killed or effectively inactivated at elevated temperatures. The mask may have a support member that is configured to extend across at least a portion of a face of a user and a heater connected to the support member.

In another aspect of the present invention, the heater may be a PTC heater. The heater may be configured to reach a temperature of at least 40 degrees Celsius or a temperature of at least 56 degrees Celsius. The heater may be configured to reach a temperature to inhibit, slow, deactivate and/or kill a virus.

In another aspect of the present invention the heater may have a first layer made of an electrically insulating material, a second electrically conductive layer, and a third resistive layer electrically connected to the second layer. The third layer may have a higher electrical resistance than the second layer and experiences a positive temperature coefficient (PTC) effect when heated. The second layer may include first and second buses spaced from each other with the resistive layer electrically connecting the first and second buses. A fourth interface layer may be directly connected to at least one of the second electrically conductive layer and the third resistive layer. The fourth layer may directly engage the support member of the mask.

In another aspect of the present invention the heater may include a base portion and a plurality of arm portions extending radially outward from the base portion. Each arm portion may include a circumferentially extending portion extending from an end of the arm portion spaced from the base portion.

In yet another aspect of the present invention the support member may be configured to extend over a mouth and nose of a user and is made of a material that permits the user to breathe through the mask.

### BRIEF DESCRIPTION OF THE DRAWINGS

The foregoing and other features and advantages of the present invention will become apparent to one skilled in the art upon consideration of the following description of the invention and the accompanying drawings, in which:

FIG. 1 is a pictorial view of a first embodiment of a heated mask constructed in accordance with the present invention being worn by a user;

FIG. 2 is a schematic view of a self-regulating heater of the heated mask of FIG. 1;

FIG. 3 is an exploded schematic view of a second embodiment of a self-regulating heater for the mask of FIG. 1;

FIG. 4 is a schematic view of another embodiment of a heated mask constructed in accordance with the present invention;

FIG. 5 is a schematic view of another embodiment of a heated mask constructed in accordance with the present invention; and

FIG. 6 is a schematic view of another embodiment of a heated mask constructed in accordance with the present invention;

### DETAILED DESCRIPTION

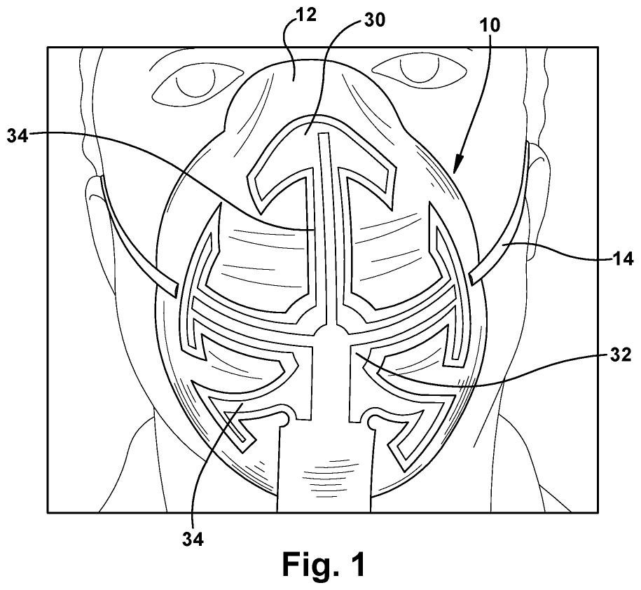

A heated mask 10 constructed in accordance with the present invention is illustrated in FIG. 1. The mask 10 includes a support member 12. The support member 12 may have a convex surface that faces away from the user and a concave surface that faces the user. The support member 12 may be made of a material that permits a user to breathe through the mask 10. An attachment member 14 may extend between first and second sides of the support member 12. The attachment member 14 may extend around a head of a user that wears the mask 10 to retain the mask 10 over the mouth and nose of the user. The support member 12 may extend across at least a portion of a face of a user when the attachment member 14 extends around the head of the user. The support member 12 can extend over the mouth and nose of the user. Although the mask 10 is shown as having one attachment member 14 that extends around a head of a user, it is contemplated that the mask may have any desired number of attachment members. The mask 10 may have attachment members that extend over the ears of the user and are connected to the first and second sides of mask.

The mask 10 includes a heater 30 connected to the support member 12. The heater 30 may engage the convex surface that faces away from the user and extend over the mouth and nose of the user when wearing the mask 10. It is contemplated that the heater 30 may engage the concave surface that faces the user. The heater 30 may be connected to the support member 12 in any desired manner, such as with an adhesive. The heater 30 may be a self-regulating heater, such as a positive temperature coefficient (PTC) heater.

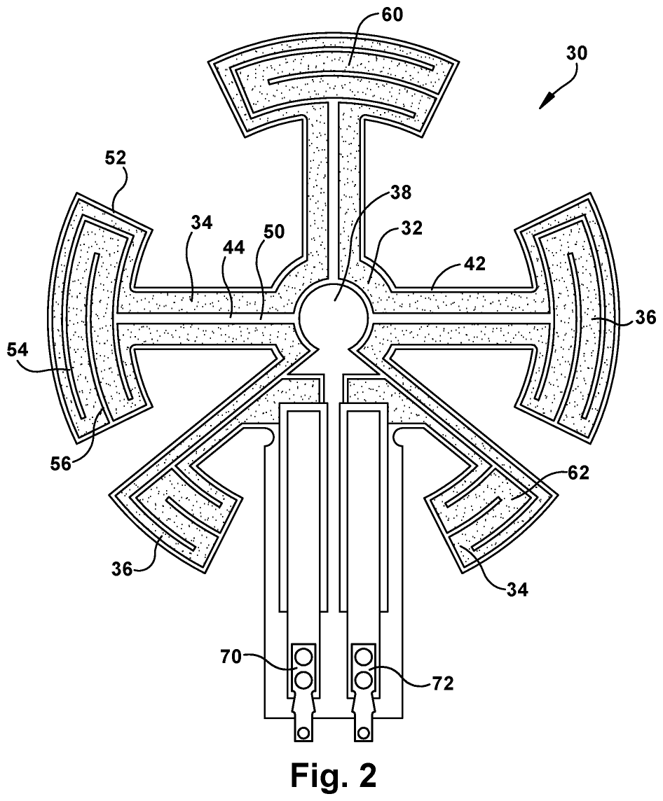

FIG. 2 illustrates an example of self-regulating heater 30 of the mask 10. The heater 30 may have a central hub or base portion 32. A plurality of arm or spoke portions 34 may extend radially from the base portion 32. The arm portions 34 are spaced from each other to permit the heater 30 to bend over curved surfaces, such as the convex or concave surface of the support member 12. Each arm portion 34 may include a circumferential portion 36 extending from an end of the arm portion spaced from the base portion 32. The heater 30 may have at least one opening 38 to increase the breathability through the heater 30. The heater 30 is shown as having one opening 38 located in the hub or base portion 32. However, it is contemplated that the heater 30 may have any number of openings 38 and the openings may extend through the arm or spoke portions 34.

The heater 30 includes a first layer or substrate 42 made of an electrically insulating material. The first layer 42 may be relatively thin and flexible. Preferably, the first layer 42 is a film substrate made of any desired electrically insulating material such as Mylar.

A second or electrically conductive layer 44 made of an electrically conductive material is connected to the first layer 42 and engages the first layer. The second layer 44 may be made of a flexible polymeric ink. The second layer 44 may be connected to the first layer 42 in any desired manner and is preferably screen printed on the first layer. The second layer 44 may be connected to the first layer 42 by close tolerance screen printing, digital printing, inkjet printing, flexographic printing, or gravure printing.

The second layer 44 may include at least two buses 50 and 52 spaced from each other. The patterns of the buses 50, 52 determine the watt density of the heater 30. The buses 50 and 52 may include finger portions 54 and 56 that extend generally between each other and parallel to each other. Although the buses 50, 52 are described as having a specific shape, it is contemplated that the buses 50, 52 may have any desired shape.

A third or resistive layer 60 that experiences a positive temperature coefficient (PTC) effect when heated is connected to the second or conductive layer 44. The third layer 60 engages the second layer 44. The third layer 60 may be connected to the second layer 44 in any desired manner and is preferably screen printed on the second layer. The third layer 60 may be connected to the second layer 44 so that the second layer is between the third layer and the first layer 42. The third layer 60 electrically connects the buses 50 and 52 of the second layer 44. The third layer 60 has a higher electrical resistance than the second layer 44. The third layer 60 generates heat when a voltage is applied across the buses 50, 52.

The third or resistive layer 60 includes a conductive carbon black filler material dispersed in a polymer that has a crystalline structure. The crystalline structure densely packs the carbon particles into a crystalline boundary so the carbon particles are close enough together to allow electrical current to flow through the polymer insulator via these carbon “chains” between the first and second buses 50 and 52. When the polymer is at normal room temperature, there are numerous carbon chains forming conductive paths through the material. Heat is produced when current flows through the polymeric device. Heating causes the temperature of the polymer to rise. As the heating continues, the temperature of the material continues to rise until it exceeds a phase transformation temperature. As the material passes through the phase transformation temperature, the densely packed crystalline polymer matrix changes to an amorphous structure. The phase change is accompanied by an expansion of the polymer. As the conductive particles move apart from each other, most of them no longer conduct current and the resistance of the heater 30 increases sharply. The heater 30 will reach a designed steady state temperature and will draw reduced amperage to maintain the steady state temperature. The heater 30 will stay “warm”, remaining in this high resistance state as long as the power is applied. Removing the power source will reverse the phase transformation and allow the carbon chains to re-form as the polymer re-crystallizes. The heater resistance returns to its original value.

A fourth or interface layer 62 is directly connected to the third layer 60 so that the fourth layer engages the third layer. The fourth layer 62 may be an adhesive or film that completely seals the heater 30. The fourth layer 62 may directly engage the support member 12. The fourth layer 62 can be a double sided adhesive and allow for the heater 30 to be assembled directly to the support member 12.

A first terminal 70 is connected to the first bus 50. A second terminal 72 is connected to the second bus 52. The terminals 70, 72 may be connected to the buses 50, 52 in any desired manner. The terminals 70, 72 connect the heater 30 to a voltage supply, such as a battery or a rechargeable battery. The fourth layer 62 may be applied after the terminals 70, 72 are connected to the buses 50, 52 or before the terminals 70, 72 are connected. If the terminals 70, 72 are connected after the fourth layer 62 is applied, openings for crimping of the terminals may be provided in the fourth layer. The terminal connections may then be sealed with a UV encapsulating material.

The heated mask 10 provides a solution that is cost effective, easy to integrate, and mass producible. The mask 10 may be a facemask, such as a surgical mask. The heater 30 can be configured for many shapes, sizes, and temperature settings. It can be configured to fit on existing masks including dust masks, surgical masks, ventilators, full face masks, and other breathing apparatuses. The heater 30 can be designed to be used with multiple voltage sources including batteries. The heater 30 can be configured to reach a temperature to inhibit, slow, deactivate and/or kill a virus. The heater 30 can be configured to reach a temperature of at least 56C to kill any possible virus. A foam ring can be added to the inside of the mask as an interface layer between the mask and skin to minimize any impact to the skin. Another aspect can be to have the heater reach a temperature of at least 40C which may deactivate the virus and allow for the heater to be closer to the skin surface without damaging the skin.

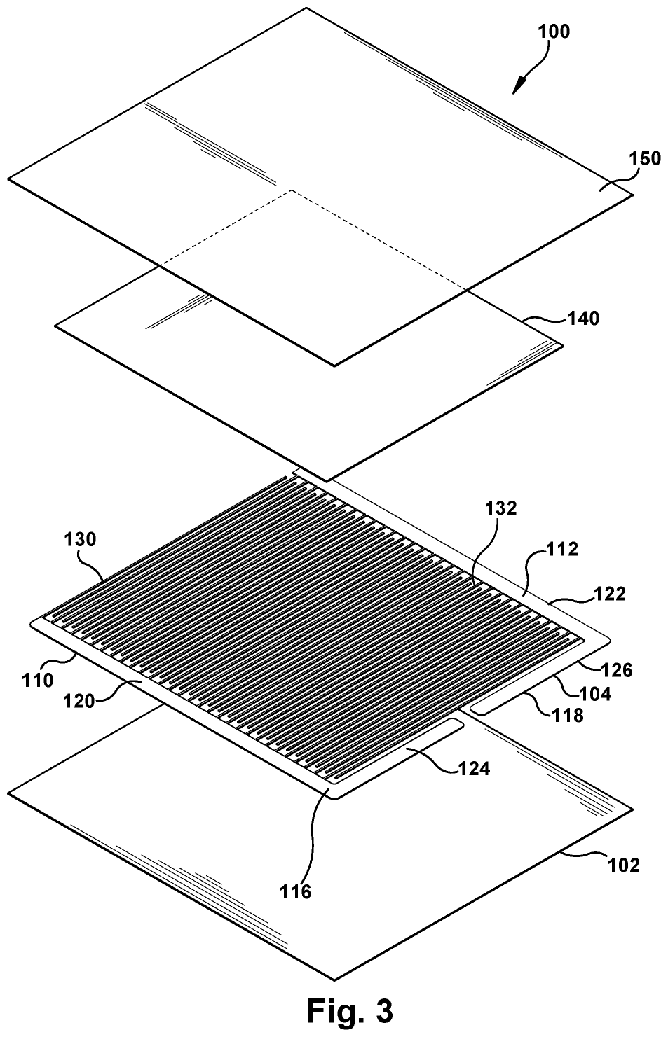

A second embodiment of a self-regulating heater 100 made in accordance with the present invention is schematically illustrated in FIG. 3. The second embodiment of the self-regulating heater 100 is generally similar to the first embodiment of the heater 30. The second embodiment of the heater 100 has a generally rectangular shape. The heater 100 includes a first layer or substrate 102 made of an electrically insulating material. The first layer 102 may be relatively thin and flexible. Preferably, the first layer 102 is a film substrate made of any desired electrically insulating material such as Mylar.

A second or electrically conductive layer 104 made of an electrically conductive material is connected to the first layer 102 and engages the first layer. The second layer 104 may be made of a flexible polymeric ink. The second layer 104 may be connected to the first layer 102 in any desired manner and is preferably screen printed on the first layer. The second layer 104 may be connected to the first layer 102 by close tolerance screen printing, digital printing, inkjet printing, flexographic printing, or gravure printing.

The second layer 104 may include at least two buses 110 and 112 spaced from each other. The patterns of the buses 110, 112 determine the watt density of the heater 100. The buses 110 and 112 may include bases 116 and 118 having a relatively large width. The bases 116 and 118 may include first portions 120 and 122 extending generally parallel to each other. Although the first portions 120 and 122 are shown extending in a straight line, it is contemplated that the first portions may extend in any desired direction.

The bases 116 and 118 may include second portions 124 and 126 extending toward each other from the first portions 120 and 122. The second portions 124 and 126 may extend perpendicular to the first portions 120 and 122. Although the second portions 124 and 126 are described as extending perpendicular to the first portions 120, 122, it is contemplated that the second portions 124 and 126 may extend in any desired direction.

The buses 110 and 112 may include finger portions 130 and 132 extending from the first portions 120 and 122. The finger portions 130 and 132 may extend generally between each other and parallel to each other and the second portions 124 and 126 of the bases 116 and 118. The finger portions 130 and 132 may have a width substantially smaller than the width of the bases 116 and 118. Although the finger portions 130, 132 are described as extending generally parallel to the second portions 124, 126, it is contemplated that the finger portions may extend in any desired direction. Although the buses 110, 112 are described as having a specific shape, it is contemplated that the buses 110, 112 may have any desired shape.

A third or resistive layer 140 that experiences a positive temperature coefficient (PTC) effect when heated is connected to the second or conductive layer 104. The third layer 140 engages the second layer 104. The third layer 140 may be connected to the second layer 104 in any desired manner and is preferably screen printed on the second layer. The third layer 140 is connected to the second layer 104 so that the second layer is between the third layer and the first layer 102. The third layer 140 electrically connects the buses 110 and 112 of the second layer 104. The third layer 140 has a higher electrical resistance than the second layer 104. The third layer 140 generates heat when a voltage is applied across the buses 110, 112 due to the PTC effect.

A fourth or interface layer 150 is directly connected to the third layer 140 so that the fourth layer engages the third layer. The fourth layer 150 may be an adhesive or film that completely seals the heater 100. The fourth layer 150 may directly engage the support member of the mask. A first terminal may be connected to the base 116. A second terminal may be connected to the base 118. The terminals may be connected to the bases 116, 118 in any desired manner, such as riveting or crimping. The terminals connect the heater 100 to a voltage supply.

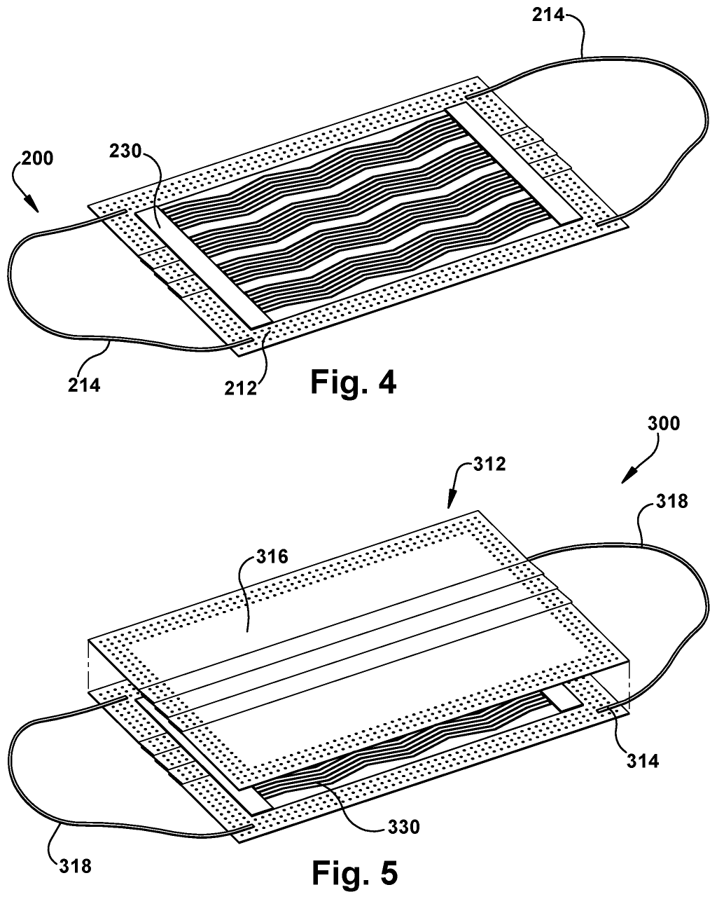

Another embodiment of a heated mask 200 made in accordance with the present invention is schematically illustrated in FIG. 4. The mask 200 includes a support member 212. The support member 212 may be a known mask made of a material that permits a user to breathe through the mask 200. Attachment members 214 may extend from first and second sides of the support member 212. The attachment members 214 may extend around the ears of a user that wears the mask 200 to retain the mask 200 over the mouth and nose of the user. The support member 212 may extend across at least a portion of a face of a user when the attachment members 214 extend around the ears of the user. The support member 212 can extend over the mouth and nose of the user. Although the mask 200 is shown as having two attachment members 214 that extend around ears of a user, it is contemplated that the mask may have any desired number of attachment members. The mask 200 may have one attachment member that extends around the head of the user and is connected to the first and second sides of mask.

The mask 200 includes a heater 230 connected to the support member 212. The heater 230 may extend over the mouth and nose of the user when wearing the mask 200. The heater 230 may be connected to the support member 212 in any desired manner, such as with an adhesive. The heater 230 may be a self-regulating heater, such as a positive temperature coefficient (PTC) heater similar to the heaters 30 and 100. The heater 230 may be removed from the mask 200. Therefore, the mask 200 may be disposed of after use and/or washed once the heater 230 is removed. The heater 230 may be connected to a support member of another mask with the adhesive so that the heater 230 may be used with more than one mask.

Another embodiment of a heated mask 300 made in accordance with the present invention is schematically illustrated in FIG. 5. The mask 300 includes a support member 312. The support member 312 may have first and second layers 314, 316 made of a material that permits a user to breathe through the mask 300. Attachment members 318 may extend from first and second sides of the support member 312. The attachment members 318 may extend around the ears of a user that wears the mask 300 to retain the mask over the mouth and nose of the user. The mask 300 includes a heater 330 connected to the support member 312. The heater 330 may be located between the layers 314, 316. The heater 330 may be connected to the layers in any desired manner, such as with an adhesive. The layers 314, 316 may be connected to each other and sealed around the edges by stitching, adhesive or glue. The heater 330 may extend over the mouth and nose of the user when wearing the mask 300. The heater 330 may be a self-regulating heater, such as a positive temperature coefficient (PTC) heater similar to the heaters 30, 100 and 230.

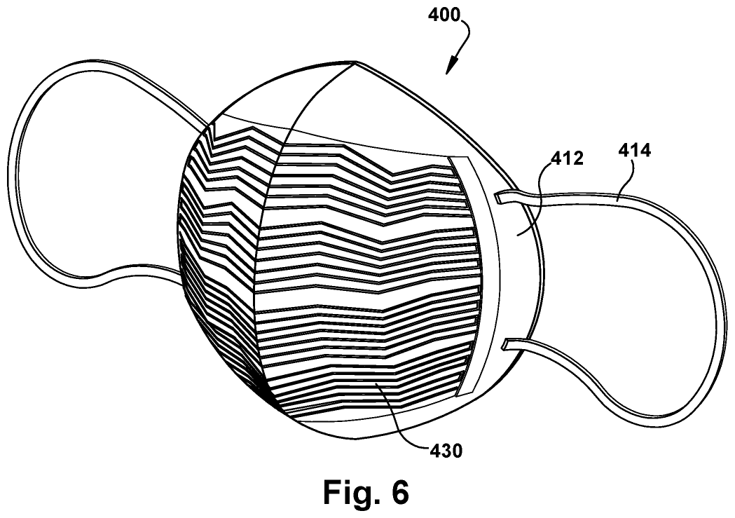

Another embodiment of a heated mask 400 made in accordance with the present invention is schematically illustrated in FIG. 6. The mask 400 includes a support member 412. The support member 412 may be a known mask made of a material that permits a user to breathe through the mask 400. Attachment members 414 may extend from first and second sides of the support member 412. The attachment members 414 may extend around the ears of a user that wears the mask 400 to retain the mask over the mouth and nose of the user. The support member 412 may extend over the mouth and nose of the user. The mask 400 includes a heater 430 connected to the support member 412. The heater 430 may extend over the mouth and nose of the user when wearing the mask 400. The heater 430 may be connected to the support member 412 in any desired manner, such as with an adhesive. The heater 430 may be a self-regulating heater, such as a positive temperature coefficient (PTC) heater similar to the heaters 30, 100, 230 and 330.

Although the heaters are shown as being on a surface of the mask that faces away from the user, it is contemplated that the heaters may be on a surface of the mask that faces toward the user. The heaters may have a controller with multiple temperature settings so that the temperature of the heaters may be adjusted. The heaters may have a temperature sensor for indicating the temperature of the heater and/or mask. It is also contemplated that the heaters and or/the masks may be sterilized by increasing the temperature of the heaters, such as 100° C. The heaters may include a thermal switch. It is also contemplated that the heaters may be operated wirelessly.

From the above description of the invention, those skilled in the art will perceive improvements, changes and modifications. Such improvements, changes, and/or modifications within the skill of the art are intended to be covered by the appended claims.

Having described the invention, the following is claimed:

1. A mask comprising a support member that is configured to extend across at least a portion of a face of a user and a heater connected to the support member.

2. A mask as set forth in claim 1 wherein the heater is a PTC heater.

3. A mask as set forth in claim 1 wherein the heater is configured to reach a temperature of at least 40 degrees Celsius.

4. A mask as set forth in claim 1 wherein the heater is configured to reach a temperature of at least 56 degrees Celsius.

5. A mask as set forth in claim 1 wherein the heater is configured to reach a temperature to inhibit, slow, deactivate and/or kill a virus.

6. A mask as set forth in claim 1 wherein the heater has a first layer made of an electrically insulating material, a second electrically conductive layer, and a third resistive layer electrically connected to the second layer, the third layer having a higher electrical resistance than the second layer, the third layer experiencing a positive temperature coefficient (PTC) effect when heated.

7. A mask as set forth in claim 6 wherein the second layer includes first and second buses spaced from each other, the resistive layer electrically connecting the first and second buses.

8. A mask as set forth in claim 6 wherein the heater includes a fourth interface layer directly connected to at least one of the second electrically conductive layer and the third resistive layer, the fourth layer directly engaging the support member of the mask.

9. A mask as set forth in claim 1 wherein the heater includes a base portion and arm portions extending radially outward from the base portion.

10. A mask as set forth in claim 9 wherein each arm portion includes a circumferentially extending portion extending from an end of the arm portion spaced from the base portion.

11. A mask as set forth in claim 1 wherein the heater includes first and second terminals for connecting the heater to a voltage source.

12. A mask as set forth in claim 1 wherein the support member is configured to extend over a mouth and nose of a user and is made of a material that permits the user to breathe through the mask.

13. A mask as set forth in claim 1 wherein the support member includes first and second layers, the heater extending between the first and second layers.

14. A mask as set forth in claim 1 wherein the heater may be removed from the support member and reused.

15. A mask as set forth in claim 1 wherein the support member has a convex surface and the heater is configured to engage the convex surface.

16. A mask as set forth in claim 1 wherein the heater is connected to a battery.

17. A mask as set forth in claim 16 wherein the batter is rechargeable.

18. A mask as set forth in claim 1 wherein the heater has multiple heat settings.

19. A mask as set forth in claim 1 wherein a temperature of the heater is adjustable.

20. A mask as set forth in claim 1 wherein the heater has openings for breathability.

21. A mask as set forth in claim 1 wherein the heater is on a surface of the mask that faces toward a user when the mask is worn by the user.

22. A mask as set forth in claim 1 wherein the heater is on a surface of the mask that faces away from a user when the mask is worn by the user.

23. A mask as set forth in claim 1 wherein the heater has a sterilization setting.

24. A mask as set forth in claim 1 wherein the heater can reach a temperature of approximately 100° C.

25. A mask as set forth in claim 1 wherein the heater has a temperature sensor.

26. A mask as set forth in claim 1 wherein the heater has a thermal switch.

27. A mask as set forth in claim 1 wherein the heater is operated wirelessly.
